(19) United States
(12) Patent Application Publication
(10) Pub. No.: US 2010/0221341 A1
(43) Pub. Date: Sep. 2, 2010

(54) Alanine-Based Peptide Hydrogels

(75) Inventors: Reinhard Schweitzer-Stenner, Drexel Hill, PA (US); Thomas J. Measey, Burlington, NJ (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Drexel University

(21) Appl. No.: 12/713,820

(22) Filed: Feb. 26, 2010

Related U.S. Application Data
(60) Provisional application No. 61/156,357, filed on Feb. 27, 2009.

(51) Int. Cl. A61K 9/00

(52) U.S. Cl. 424/484

(57) ABSTRACT

The present invention includes a method of preparing a hydrogel of a selected viscosity. The present invention further includes a method of promoting controlled release of a biomolecule into a medium using a hydrogel of a selected viscosity.

### CROSS-REFERENCE TO RELATED APPLICATION

This application claims priority under 35 U.S.C. §119(e) to U.S. Provisional Application No. 61/156,357, filed Feb. 27, 2009, which application is incorporated by reference herein in its entirety.

### STATEMENT REGARDING FEDERALLY SPONSORED RESEARCH OR DEVELOPMENT

This invention was made with government support under Grant No. CHEM 0804492 awarded by National Science Foundation. The government has certain rights in the invention.

### BACKGROUND OF THE INVENTION

The self-assembly of biomolecules, such as peptides and proteins, has become an important issue in biomedical, biotechnological, and material research. The aggregation of peptides may be considered as a non-covalent form of a polymerization process. The biomedical relevance of peptide aggregation results from the fact that different disorders such as spongiform encephalopathy, Alzheimer's disease, Parkinson's disease, and Huntington's disease are associated with the aggregation and subsequent fibril formation of naturally unfolded or misfolded peptides and proteins (Kelly, 1998, Curr. Opin. Struct. Biol. 8:101; Dobson, 1999, Trends Biochem. Sci. 24:329; Bellotti et al., 1999, Cell. Mol. Life. Sci. 55:977; Rochet & Landsbury, 2000, Curr. Opin. Struct. Biol. 10:60).

With respect to biotechnology, the self-assembly process allows the generation of material with incorporated biofunctionality and potential biocompatibility (Rajagopal & Schneider, 2004, Curr. Opinion Struct. Biol. 14:480). Self-assembled molecules offer the possibility of creating new supramolecular architectures such as ribbons, nanotubes, and monolayers exhibiting a nanoscale order (Holmes et al., 2000, Proc. Natl. Acad. Sci. U.S.A. 97:6728; Vauthey et al., 2002, Proc. Natl. Acad. Sci. U.S.A. 99:5355).

Self-aggregating peptides are often hydrophobic, with charged residues added so that they may dissolve at low concentrations (such as Ac-KYA₁₃K-NH₂ or D₂A₁₀K₂; Warras et al., 2000, J. Am. Chem. Soc. 122:1789-1795; Nguyen et al., 2004, Protein Sci. 13:2909-2924; Perutz et al., 2002, Proc. Natl. Sci. U.S.A. 99:5596-5600). Peptides with alternating hydrophilic and hydrophobic residues (such as VKVKVK or KFEFK segments) are also used, since they are amphipathic with respect to β-strand or β-sheet structure (Schneider et al., 2002, J. Am. Chem. Soc. 124:15030-15037; Caplan et al., 2000, Biomacromolecules 1:627-631). Other types of peptides with antibacterial and hemolytic activity have a mixture of hydrophilic and hydrophobic groups and prefer helical structures in membrane environments (Blondelle et al., 1992, Biochemistry 31:12688-12694; Pathak et al., 1995, Proteins: Struct., Funct., Genet. 22:182-186). The helical wheel projections of these peptides show a clustering of equally charged groups and hydrophobic groups, respectively. Alanine-based peptides doped with some charged residues generally form short a-helices in solution if the number of residues exceeds a certain threshold value (Scholtz et al., 1991, Proc. Natl. Acad. Sci. U.S.A. 88:2854-2860).

The term “hydrogel” (also called aquagel) refers to a network of oligomers or polymer chains that are water-insoluble, sometimes found as a colloidal gel in which water is the dispersion medium. Hydrogels are natural or synthetic polymers that show superabsorbent properties (having even over 99% water). Hydrogels also possess a degree of flexibility very similar to natural tissue, due to their significant water content. Common uses for hydrogels include: scaffolds in tissue engineering (agarose, methylcellulose, hylaronan, and other naturally derived polymers); environmentally sensitive hydrogel; sustained-release delivery systems; absorbing material; contact lenses; granules for holding soil moisture in arid areas; dressings for healing of burn or other hard-to-heal wounds; reservoirs in topical drug delivery; and ingredients (e.g. polyvinyl alcohol, sodium polyacrylate, acrylate polymers and copolymers with an abundance of hydrophilic groups).

There is a great need in identifying novel compounds that form stable hydrogels under reproducible and controlled conditions. Such hydrogels could be used for the transport and controlled release of biomolecules in biological systems. The present invention fulfills this need.

### SUMMARY OF THE INVENTION

The invention includes a method of preparing a hydrogel of a defined viscosity. The method comprises dissolving a peptide in an aqueous solution, wherein the peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4), to generate a first solution. The first solution is then equilibrated at a given temperature for a given period of time. An amount of salt ingredient is then added to the first solution, to generate a second solution, wherein the second solution comprises the hydrogel of the defined viscosity.

In one embodiment, the concentration of the peptide in the second solution ranges from about 1 mg/mL to about 20 mg/mL. In another embodiment, the concentration of the peptide in the second solution ranges from about 5 mg/mL to about 10 mg/mL. In yet another embodiment, the given period of time ranges from about 1 minute to about 48 hours. In yet another embodiment, the given period of time is about 24 hours. In yet another embodiment, the given temperature is about 25° C. In yet another embodiment, the salt ingredient comprises sodium chloride. In yet another embodiment, the concentration of the salt ingredient in the second solution ranges from about 0.5 M to about 10 M. In yet another embodiment, the defined viscosity ranges from about 500 cP to about 3000 cP for a shear rate of 1/s. In yet another embodiment, the defined viscosity ranges from about 800 cP to about 2500 cP for a shear rate of 1/s.

The invention further includes a method of promoting controlled release of a biomolecule into a medium using a hydrogel of a defined viscosity. In one aspect, the method comprises preparing a first solution comprising the biomolecule, and then dissolving a peptide in the first solution, wherein the peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4), to generate a second solution. In another aspect, the method comprises preparing a first solution comprising a peptide, wherein the peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4), and then dissolving the biomolecule in the first solution, to generate a second solution. The second solution is then equilibrated at a given temperature for a given period of time. An amount of salt ingredient is then added to the second solution, to generate a third solution, wherein the third solution comprises the hydrogel of the defined viscosity. The third solution is then contacted with the medium, whereby the biomolecule undergoes controlled release into the medium.

In one embodiment, the concentration of the peptide in the third solution ranges from about 1 mg/mL to about 20 mg/mL. In another embodiment, the concentration of the peptide in the third solution ranges from about 5 mg/mL to about 10 mg/mL. In yet another embodiment, the given period of time ranges from about 1 minute to about 48 hours. In yet another embodiment, the given period of time is about 24 hours. In yet another embodiment, the given temperature is about 25° C. In yet another embodiment, the salt ingredient comprises sodium chloride. In yet another embodiment, the concentration of the salt ingredient in the third solution ranges from about 0.5 M to about 10 M. In yet another embodiment, the defined viscosity ranges from about 500 cP to about 3000 cP for a shear rate of 1/s. In yet another embodiment, the defined viscosity ranges from about 800 cP to about 2500 cP for a shear rate of 1/s.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the isotropic Raman, anisotropic Raman, FTIR, and VCD spectra of the amide I′ band of Ac-(AAKA)₄-NH₂ measured at room temperature.

FIG. 2 depicts mechanisms of association of peptide strands.

FIG. 3 illustrates the isotropic and anisotropic visible Raman, and FTIR amide I′ band spectra of the aggregated state of Ac-(AAKA)₃-NH₂.

FIG. 4 illustrates the UV-CD spectra of Ac-(AAKA)₄-NH₂ after dissolution in D₂O, as a function of time.

FIG. 5 illustrates the UV-CD kinetics of Ac-(AAKA)₄-NH₂ monitored at 198 nm and 215 nm.

FIG. 6 illustrates the FT-IR kinetics of the amide II and amide II′ band for Ac-(AAKA)₄-NH₂.

FIG. 7 illustrates the UV-CD spectra of 3.5 mM Ac-(AAOA)₄-NH₂ (SEQ ID NO:6) and Ac-(AAKA)₃-NH₂.

FIG. 8 illustrates the temperature-dependent UV-CD spectra of 3.5 mM Ac-(AAKA)₄-NH₂ in D₂O (pH=2.7). The arrows indicate the direction of temperature increase. The inset displays the change of the Ac measured at 214.5 nm as a function of temperature.

FIG. 9, comprising FIGS. 9A and 9B, illustrate REMD (replica exchange molecular dynamics) convergence test. FIG. 9A illustrates the replica exchange history of one of the REMD trajectories for the trimer simulation. Other trajectories showed similar behavior. FIG. 9B illustrates the fraction of extended structures as a function of REMD simulation time at 300 K for the trimer simulation. The solid line is raw data, and the gray line is the block average for every 200 data points.

FIG. 10 illustrates the reference structure for the rmsd calculation obtained from the clustering analysis. The dashed lines represent hydrogen bonds.

FIG. 11 illustrates the distribution of the fraction of trimer, dimer+monomer, and three monomers at different temperatures. Conformers above 400 K are highly disordered, and this distinction has no meaning.

FIG. 12 illustrates the temperature-dependent FT-IR spectra of 3.5 mM Ac-(AAKA)₄-NH₂ in D₂O (pH=2.7). Legends show temperatures in ° C.

PHIP/ 796261.4

Attorney Docket No 046528-6008-00-US [448727]

FIG. 13, comprising FIGS. 13A-13F, illustrates the backbone hydrogen bond network for the top six most populated clusters. Schematic plots of the backbone hydrogen bond network for the representative conformers of the top six most populated clusters, ordered from the most populated (FIG. 13A) to the least populated (FIG. 13F). The arrows represent the backbone hydrogen bond with the direction pointing from carbonyl oxygen to amino hydrogen, while the vertical bars indicate the repeat unit.

FIG. 14 illustrates the UV-CD spectra of 3.5 mM and 7 mM (AAKA)₄ (SEQ ID NO:1) as a function of NaCl concentration.

FIG. 15 illustrates a hydrogel formed by the addition of NaCl to a freshly prepared solution of (AAKA)₄.

FIG. 16 illustrates rheology measurements made with a 5 mg/mL solution of (AAKA)₄ in 0.5 M NaCl and 1.0 M NaCl.

FIG. 17 illustrates rheology measurements made with a 5 mg/mL solution of (AAKA)₄ in 0.0 M NaCl, 1.0 M NaCl and 2.0 M NaCl.

FIG. 18 illustrates rheology measurements made with a 5 mg/mL solution of (AAKA)₄ in 1 M NaCl, and a 5 mg/mL solution of (AAKA)₄ in 1.0 M NaCl.

FIG. 19 illustrates rheology measurements made with a 5 mg/mL solution of (AAKA)₄ where NaCl was added at time zero with a 5 mg/mL solution of (AAKA)₄ where NaCl was added ˜24 hours after the peptide solution was prepared. In all cases the final concentration of NaCl was 2.0 M.

FIG. 20, comprising FIGS. 20A-20C, illustrates the time-dependent atomic force analysis of a 7 mM solution of (AAKA)₄. Time increases from left to right.

FIG. 21, comprising FIGS. 21A-21C, illustrates atomic force analysis of the hydrogel formed from a 7 mM solution of (AAKA)₄ (FIGS. 21A-21B) and from a 7 mM solution of (AAKA)₄ (FIG. 21C).

FIG. 22 illustrates the release profile of equine cytochrome c from (AAKA)₄ hydrogel, prepared with a final NaCl concentration of 1.0 M. Diffusion coefficients were obtained by plotting the normalized concentration of cytochrome c released versus (time)^{0.5}.

FIG. 23, comprising FIG. 23A and FIG. 23B, depicts aggregation of the peptide (FEFEFKFK)₂ (SEQ ID NO:7) triggered by the release of CaCl₂ from liposomes, by light (FIG. 23A) or by temperature (FIG. 23B).

FIG. 24 illustrates simulated amide I band profiles of the IR, isotropic Raman, anisotropic Raman and VCD spectra of different oligomers of Ac-(AAKA)₄-NH₂. The number of strands in the considered sheet monomer is indicated.

FIG. 25 illustrates changes in the amide I band profile of the (AAKA)₄ peptide, as a function of time.

FIG. 26 illustrates a MD simulation for the aggregation of the (AAKA)₄ peptide.

FIG. 27 is non-limiting model of the (AAKA)₄ peptide in an alpha-helix structure.

FIG. 28 illustrates the VCD spectrum of a 1-year-old sample of 5 mg/mL (3.5 mM) (AAKA)₄, showing the amide I′ band. The negative couplet was indicative of a monomeric poly-L-proline II (PPII) secondary structure. The addition of NaCl did not result in hydrogel formation, suggesting that β-like structure is required for hydrogel formation. As a comparison, the VCD spectrum is also shown for monomeric (AAKA)₂ (SEQ ID NO:8), which was shown to contain ˜40% PPII structure (Measey & Schweitzer-Stenner, 2006, J. Raman Spectr. 37, 248-254).

FIG. 29 illustrates the absorbance of (AAKA)₄ (5 mg/mL, 3.5 mM) in D₂O as a function of NaCl concentration, highlighting the FTIR amide I′ band.

FIG. 30 depicts the absorbance of (AAKA)₄ (5 mg/mL, 3.5 mM) in D₂O as a function of time, highlighting the FTIR amide I′ band.

FIG. 31, comprising FIGS. 31A-31D, illustrates the atomic force microscopy (AFM) for the (AAKA)₄ peptide. FIG. 31 A illustrates the experiment where peptide was 5 mg/mL, and incubation was for 10 min. FIG. 31 B illustrates the experiment where peptide was 5 mg/mL, and incubation was for 30 min. FIG. 31C illustrates the experiment where peptide was 5 mg/mL in 0.5 M NaCl, and incubation was for 20 min. FIG. 31 D illustrates the experiment where peptide was 5 mg/mL in 0.5 M NaCl, and incubation was for 30 min.

### DETAILED DESCRIPTION OF THE INVENTION

The present invention relates to the discovery that the viscosity of a hydrogel prepared with the peptides contemplated within the invention may be controlled. Varying the time between the dissolution of the peptides contemplated within the invention and the addition of salt to the resulting solution may be used to generate a hydrogel of a defined viscosity.

In one aspect, the invention provides includes a method of preparing a hydrogel of a defined viscosity, using the peptides contemplated within the invention. In another aspect, the invention provides a method of promoting controlled release of a biomolecule into a medium using a hydrogel of a defined, using the peptides contemplated within the invention.

### Definitions

As used herein, each of the following terms has the meaning associated with it in this section.

As used herein, unless defined otherwise, all technical and scientific terms generally have the same meaning as commonly understood by one of ordinary skill in the art to which this invention belongs. Generally, the nomenclature used herein and the laboratory procedures in cell culture, molecular genetics, organic chemistry, and peptide chemistry are those well known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used.

As used herein, “about” when referring to a measurable value such as an amount, a temporal duration, and the like, is meant to encompass variations of ±20% or ±10%, more preferably +5%, even more preferably ±1%, and still more preferably ±0.1% from the specified value, as such variations are appropriate to perform the disclosed methods.

As used herein, the term “UV-CD” means ultraviolet-circular dichroism, the term “VCD” means vibrational circular dichroism, and the term “FT-IR” means Fourier Transform Infrared.

As used herein, the term “hydrogel” or “aquagel” refers to a network of oligomers or polymer chains that are water-insoluble, sometimes found as a colloidal gel in which water is the dispersion medium.

As used herein, the term “salt ingredient” refers to one or more salts that may be added simultaneously, sequentially or separately to a solution. The concentration of the salt ingredient in the resulting solution is given by the sum of the molarities of the one or more salts involved.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, the polypeptides include natural peptides, recombinant peptides, synthetic peptides or a combination thereof. A peptide that is not cyclic has an N-terminus and a C-terminus. The N-terminus has an amino group, which may be free (i.e., as a NH₂ group) or appropriately protected (for example, with a BOC or a Fmoc group). The C-terminus has a carboxylic group, which may be free (i.e., as a COOH group) or appropriately protected (for example, as a benzyl or a methyl ester). A cyclic peptide does not necessarily have free N- or C-termini, since they are covalently bonded through an amide bond to form the cyclic structure. The term “peptide bond” means a covalent amide linkage formed by loss of a molecule of water between the carboxyl group of one amino acid and the amino group of a second amino acid.

As used herein, amino acids are represented by the full name thereof, by the three letter code corresponding thereto, or by the one-letter code corresponding thereto, as indicated below:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

Preparation of a Hydrogel with a Defined Viscosity

The present invention includes a method of preparing a hydrogel with a defined viscosity. In one aspect, the viscosity of the hydrogel may be manipulated by varying the delay time between the preparation of a solution of a peptide useful within the invention and the addition of a salt ingredient to the solution of the peptide.

The relationship between the viscosity of the resulting hydrogel and the delay time in question may be established by conducting parallel experiments, where hydrogels are prepared under controlled circumstances with varying delay times between the preparation of the peptide solution and the addition of a salt ingredient to the peptide solution. The viscosity of the resulting hydrogels may be measured, and an empirical correlation between delay times and viscosity may be derived. Viscosity may also be influenced by other controllable factors, such as nature of peptide, concentration of peptide solution, temperature of solution, nature of salt ingredient and concentration of salt ingredient.

In a non-limiting example, a peptide useful within the invention is dissolved in an aqueous solution. The peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4). This procedure generates a first solution. The first solution is equilibrated at a given temperature for a given period of time, and then an amount of salt ingredient is added to the first solution. A second solution is thus generated, comprising a hydrogel of a defined viscosity.

Controlled Release of a Biomolecule using a Hydrogel of a Defined Viscosity

The present invention includes a method of promoting controlled release of a biomolecule into a medium using a hydrogel of a defined viscosity. The viscosity of the hydrogel may influence the rate of release of the biomolecule into the medium. In a non-limiting example, a first solution comprising the biomolecule is prepared, and then a peptide useful within the invention is dissolved in the first solution. Alternatively, the peptide useful in the invention is dissolved in a first solution, and then the biomolecule is dissolved in the first solution. The peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4). This generates a second solution. The second solution is equilibrated at a given temperature for a given period of time, and an amount of salt ingredient is added to the second solution, to generate a third solution. The third solution comprises the hydrogel of a defined viscosity. The third solution is then contacted with the medium, wherein the biomolecule is released into the medium.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only, and the invention is not limited to these Examples, but rather encompasses all variations that are evident as a result of the teachings provided herein.

### Materials and Methods

### Compounds and Instrumentation

The peptides useful within the invention were prepared by synthetic methods known to those in the art, such as, but not limited to, solid-phase peptide synthesis.

For the FT-IR (Fourier transform infrared), CD (circular dichroism), and Raman measurements of Ac-(AAKA)₄-NH₂, unless otherwise described, the peptide was dissolved in acidic D₂O (pD=1.4) at a concentration of ˜15 mg/mL (˜10.5 mM), where the D₂O was made acidic by the addition of DCl (Sigma-Aldrich). The peptide formed a hydrogel shortly after dissolution in D₂O, and the measurements were acquired on the hydrogel. The measurements for the gelated state of the 12-mer, Ac-(AAKA)₃-NH₂, were carried out similarly, at a concentration of ˜27 mg/mL in D₂O (pD=1.4). For the ECD measurements, Ac-(AAKA)₃-NH₂ and (AAKA)₄ were dissolved in acidic D₂O at concentrations of 2.4 mg/mL and 1 mg/mL, respectively, and the samples were measured at room temperature.

The IR and VCD spectra were recorded with a Chiral IRTM Fourier Transform VCD spectrometer from Bio Tools. The sample was placed into a cell with a pathlength of 42.5 rim. The spectral resolution was 4 cm⁻¹. The absorptivity values of these spectra were converted to molar extinction (M⁻¹.cm⁻¹.residue⁻¹) by using Beer-Lambert's law.

The Raman spectra were obtained with the 442 nm (40 mW) excitation from a HeCd laser (Model IK 4601R-E, Kommon Electrics, USA) and recorded with a RM 100 Remishaw confocal microscope. The UV ECD spectra in the wavelength range of 180-250 nm of peptides were measured with a JASCO J-810 spectrapolarimeter in a 0.1 mm quartz cell with 0.05 nm resolution. Samples were placed in a nitrogen purged JASCO CD module. The temperature at the cuvette was controlled by means of a Peltier-type heating system (accuracy ±1° C.) set to 20° C. The spectra were obtained by averaging ten scans and were collected as ellipticity as a function of wavelength, and converted to units of molar absorptivities via the equation:

Δε=θ/[32980.l.c]

where θ is the ellipticity in [mdeg], l is the pathlength of the cuvette in [cm] and c is the concentration in [M].

The IR and Raman spectra were treated with the program MULTIFIT, which was also used for determining the wavenumber of band peaks. All spectra were finally produced with the SIGMA Plot 6.0 software.

Replica exchange molecular dynamics (REMD) simulation was performed as described in Jang et al., 2009, J. Phys. Chem. B, 113:6054-6061.

### Characterization of Alanine-Based Peptides:

### IR, Raman and ECD Spectra of Ac-(AAKA)₄-NH₂

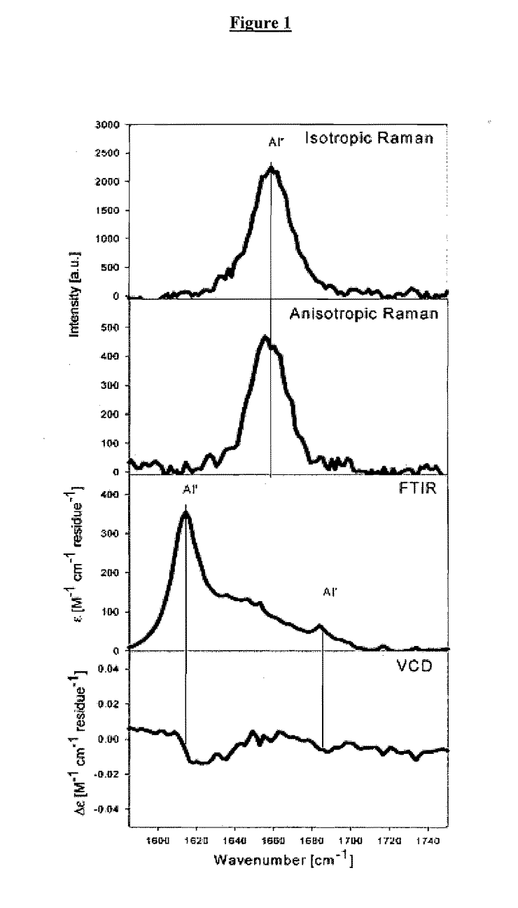

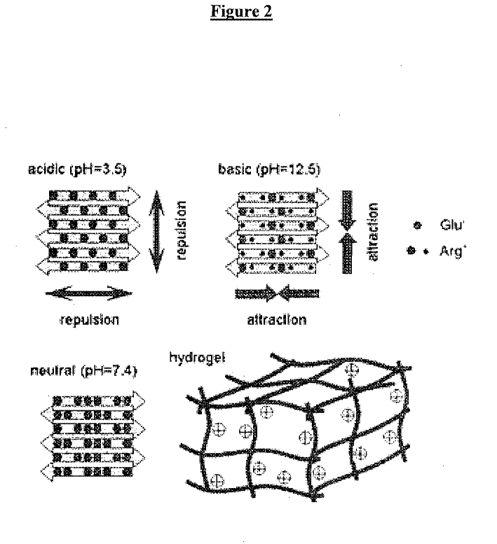

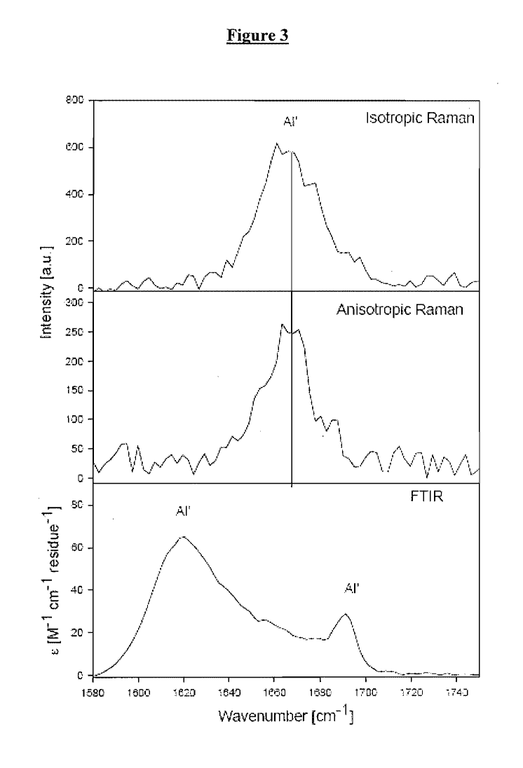

The IR, isotropic and anisotropic Raman, and VCD spectra of the amide I′ region of the Ac-(AAKA)₄-NH₂ hydrogel are illustrated in FIG. 1. The IR-spectrum exhibited a strong, asymmetric band at 1616 cm⁻¹ with its high wavenumber wing extending over a very broad spectral region. A much smaller peak was observed at 1684 cm⁻¹. The VCD spectrum displayed two weak negative peaks, one at 1620 cm⁻¹, and an even less-pronounced peak at 1685 cm⁻¹. Both the IR and the rather weak VCD spectra are associated with an antiparallel β-sheet conformation (Keiderling, T. In “Circular Dichroism: Principles and Applications”; Bernova, N., Nakanishi, K., Woody, R. W., Eds.; Wiley: New York, 2000, pp. 621-666). A very strong interstrand excitonic coupling gives rise to the observed band splitting. The “continuum” between the two IR bands stems from the distribution of excitonic states typical for a not perfectly regular β-strand (Bour & Keiderling, 2004, J. Mol. Struct.: THEOCHEM 675:95-105). The large splitting may indicate the absence of significant twisting.

The Raman spectra in FIG. 1 exhibited a broad band at approximately 1655 cm⁻¹. A fit of a Voigtian profile to the isotropic and anisotropic Raman band revealed a slight non-coincidence (-2.0 cm⁻¹) between the respective peak positions with the isotropic band at higher wavenumbers. This indicated a distribution of excitonic states rather than the single band expected for an ideal β-sheet (Krimm & Bandekar, 1986, Adv. Protein Chem. 38:181). A preliminary simulation of the IR and Raman spectra of β-hairpin with a type I′-β turn was performed, on the basis of the excitonic coupling model (Measey & Schweitzer-Stenner, 2005, Chem. Phys. Lett. 408:123- 127), and this simulation nearly exactly yielded the observed non-coincidence.

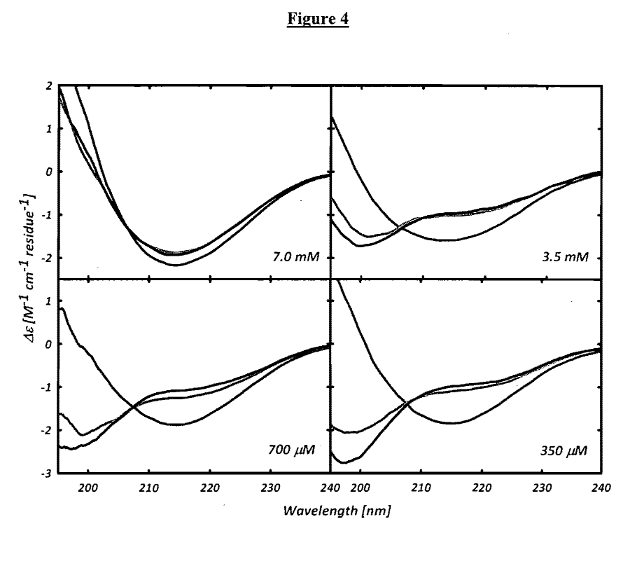

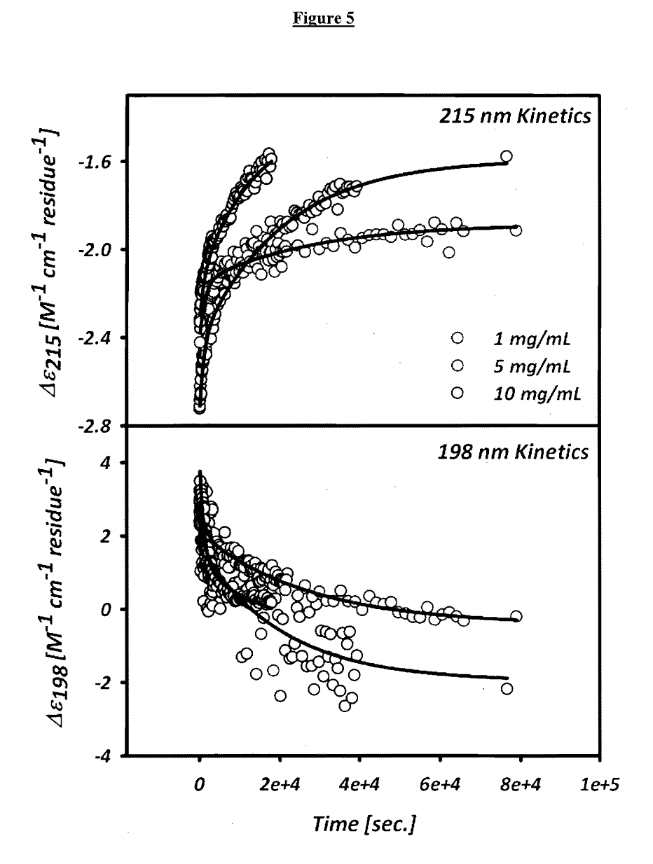

Electronic circular dichroism (ECD) measurements were performed at room temperature and a lower peptide concentration (1-2 mg/mL) where aggregation did not take place (FIG. 4). The ECD of the 16-mer was indicative of an a-helical conformation mixed with some PPII. Aggregation may involve an α→β transition at a higher concentration, resembling the behavior of prion proteins.

### Characterization of Alanine-Based Peptides.

### IR, Raman and ECD Spectra of Ac-(AAKA)₃-NH₂

Measurements were also performed on the gelated state of Ac-(AAKA)₃-NH₂. IR and Raman spectra are illustrated in FIG. 3. These spectra were consistent with a β-sheet structure, but compared with the spectra of Ac-(AAKA)₄-NH₂ all bands were substantially broadened and the high wavenumber band in the IR spectrum was more intense. This suggests a more inhomogeneous solution and structural differences between the formed β-sheets.

A significant fraction of non-aggregated peptides was suggested by the isotropic Raman intensity between 1660 and 1670 cm⁻¹. The respective ECD spectrum measured at room temperature (FIG. 4) suggested a disordered peptide, with a mixture of PPII, β-strand, and a-helix-like conformations.

The formation of a β-sheet-containing gel is unusual for an amphipathic peptide with its charged groups clustering in the helical wheel, for which the formation of a helical structure with the tendency to layer on the aqueous surface would be expected. The positive charges on lysine would in principle exclude the possibility of regular multistrand sheet layers solely formed by interpeptide hydrogen bonds. The hydrophobic interaction between the alanine side chain could give rise to a β-hairpin like conformation with the lysine pointing outside and the inside occupied by alanines. However, a hairpin structure alone cannot explain the gel formation and the IR-splitting observed experimentally. Hence, further stacking and in-plane aggregation should be necessary. This could take place by hydrogen bonding between lysine and peptide carbonyls of different hairpin structures. A possible reason for the peptide's sheet propensity may be associated with the finding that AAKA exhibits more β-strand character than tetraalanine, possibly because lysine prevents an efficient hydration of the protein backbone.

### Example 1

### Conformational Instability of Ac-(AAKA)₄-NH₂: UV-CD Spectra.

The UV-CD spectra of Ac-(AAKA)₄-NH₂ was acquired at various times after dissolution in deuterated water (D₂O). The results are illustrated in FIG. 4. Initially a significant percentage of β-structure was observed, and in a time-dependent manner more flexible PPII-like conformations were observed.

### Example 2

### Conformational Instability of Ac-(AAKA)₄-NH₇: UV-CD Kinetics.

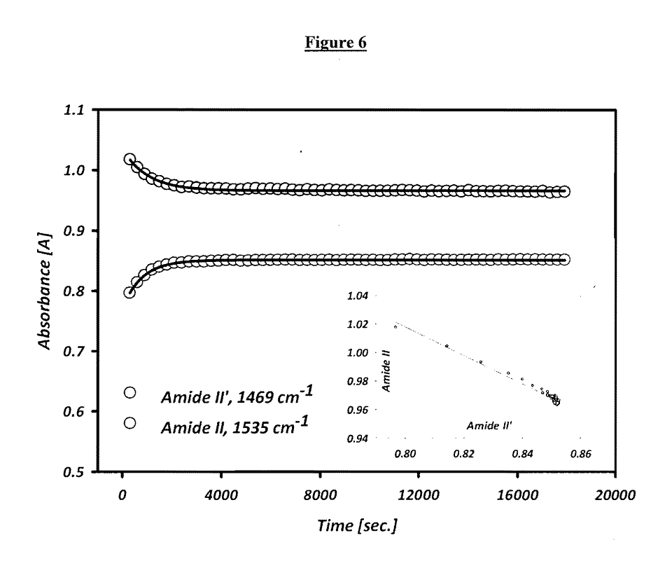

The UV-CD kinetics of Ac-(AAKA)₄-NH₂, in terms of the readings at 198 nm and 215 nm as a function of time, were monitored. The results are illustrated in FIG. 5. The plots were fit to bi-exponential functions, reflecting a fast phase (˜18 minutes) and a slow phase (>300 minutes). The fast phase may reflect the kinetics of hydration/dissolution of the peptide. This interpretation is consistent with the kinetics of the amide II′/amide II band, which shows a linear correlation between the intensity of each, with the amide II′ increasing at the expense of the amide II band. The slow phase may be associated with a conformational rearrangement or change.

### Example 3

### Conformational Instability of Ac-(AAKA)₄-NH₇: FT-IR Kinetics.

The FT-IR kinetics of the amide II and amide II′ band for Ac-(AAKA)₄-NH₂ was monitored, and the results are illustrated in FIG. 6. The linear correlation observed suggested that the fast phase observed in the UV-CD kinetics (Example 2) was due to hydration.

### Comparative Example 1

### UV-CD Spectra of Ac-(AAOA)₄-NH₂ and Ac-(AAKA)₃-NH₂.

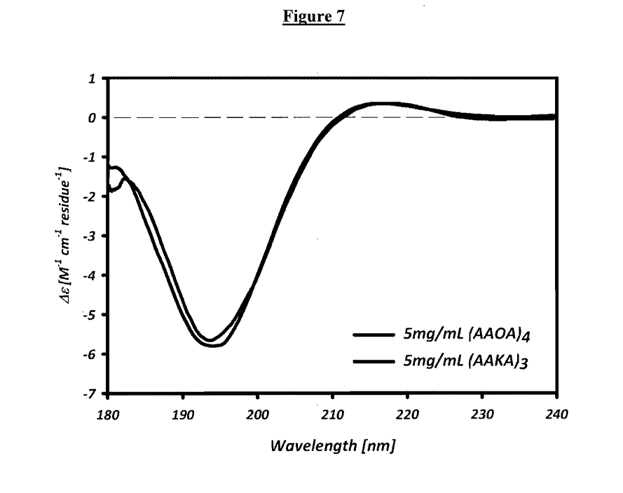

The UV-CD spectra of 3.5 mM Ac-(AAOA)₄-NH₂ (SEQ ID NO:6) and Ac-(AAKA)₃-NH₂ (SEQ ID NO:5) were acquired, and are illustrated in FIG. 7. Both spectra showed a predominantly PPII-like conformation.

### Example 4

### Temperature-Dependent UV-CD Spectra of Ac-(AAKA)₄-NH₂.

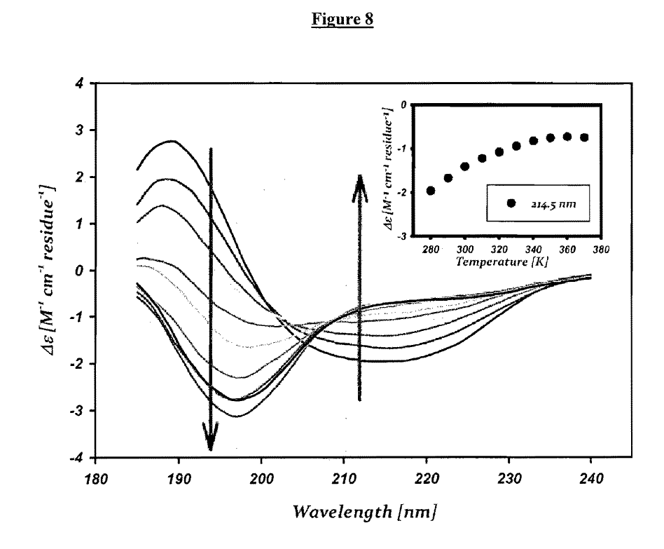

The temperature-dependent UV-CD spectra of 3.5 mM Ac-(AAKA)₄-NH₂ measured at acid pH is illustrated in FIG. 8. The hydrogel may not have been formed due to the low ionic strength of the solution. However, the room temperature spectrum clearly indicated the formation of soluble β-sheets. At high temperatures the aggregates melted into a statistical coil ensemble with a substantial fraction sampling the PPII region of the Ramachandran plot. The inset of FIG. 8 illustrates the change of the Δε measured at 214.5 nm as a function of temperature, reflecting the decrease of the β-sheet content with increasing temperature.

Two different modes were found in the relationship between the n-sheet content and temperature. The β-sheet content, as evaluated by Δε, increased linearly with temperatures below 300 K and remained almost constant after 350 K. The transition temperature between these two modes was around 320 K, as is defined by the intersection between the two straight lines, fitting the initial and final behaviors. The CD spectra did not exhibit an isodichroic point which would be indicative of a two-state transition.

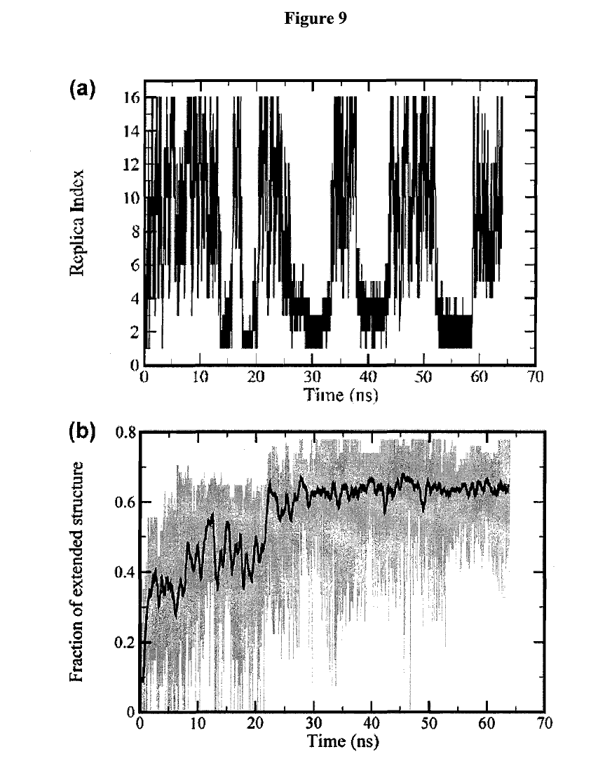

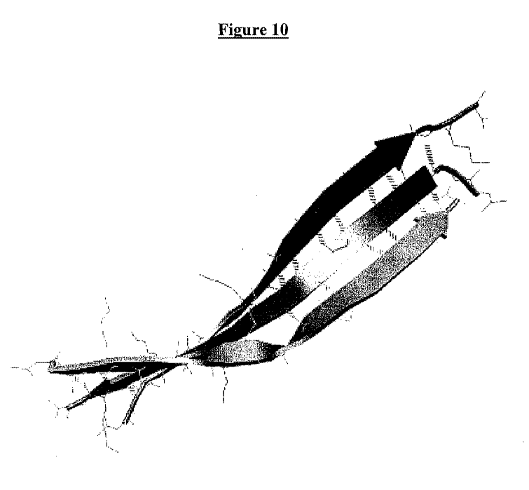

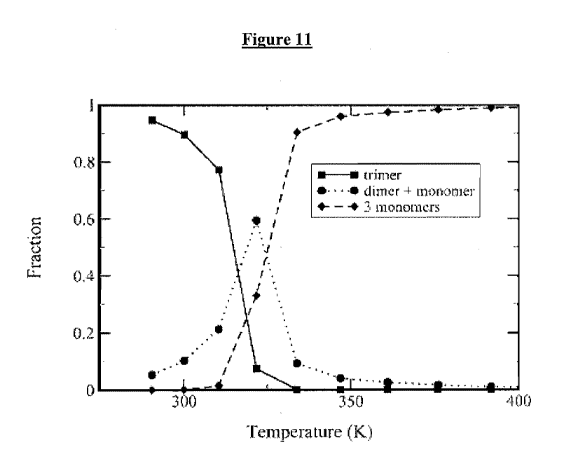

The simulation trajectory of (AAKA)₄ was traced and its replica exchange history was monitored (Jang et al., 2009, J. Phys. Chem. B, 113:6054-6061). In FIG. 9A, the replica exchange history of one trajectory is illustrated for the last 33 ns. This profile revealed how a given trajectory experiences different temperatures as replica exchange is performed, and was used as one of the measures of the quality of the REMD simulation. The trajectory visited all replica indices, i.e., the entire temperature space specified in REMD, during the REMD simulation, suggesting that reasonable stochastic trajectory exchange had been archived in the current simulation. In FIG. 9B, the fraction of extended structures as a function of REMD simulation time at 300 K for the trimer was used to measure the REMD convergence. The extended fraction remained roughly the same after 25 ns. The other secondary structures also showed similar behaviors, indicating that the REMD simulation was reasonably converged.

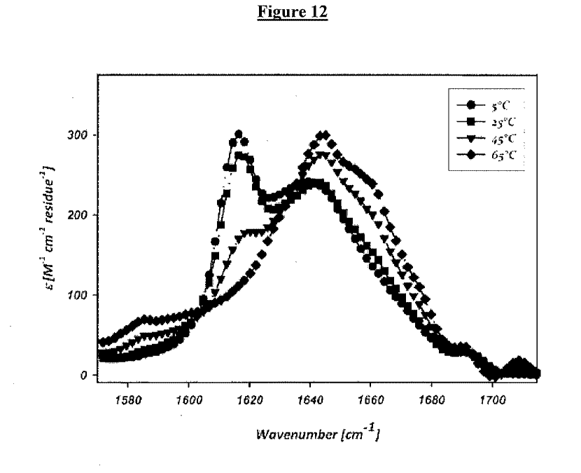

The simulation was consistent with antiparallel β-sheet formation, because the major conformers obtained by the clustering analysis also depicted an antiparallel β-strand at 300 K. The selected reference conformer contained three well-defined antiparallel β-strands (FIG. 10). Clustering analysis was performed on the results obtained by using the implicit water model at 300 K, using a 3.5 Å rmsd as the clustering criterion. The results showed that the first three clusters with relative populations of 25%, 4%, and 1.5% are trimer aggregates. In fact, about 99% of the conformers at 300 K were trimer aggregates. This suggested that a predominant fraction of (AAKA)₄ has aggregated at 300 K, consistent with experiments.

FIG. 11 illustrates the respective fractions of trimers, dimers, and monomers, as a function of temperature. FIG. 11 suggests that the trimer fraction was dominant at room temperature and melted into a mixture of dimers and monomers at ˜315 K. Above 330 K, the monomer became the dominant species, and the respective fractions of aggregates became negligible. This result agreed with the experimental UV-CD data of FIG. 8 and the FTIR data of FIG. 12.

FIG. 12 illustrates the temperature-dependent amide I′ IR spectra of Ac-(AAKA)₄-NH₂ in D₂O. The intense peak at ˜1615 cm⁻¹, as well as the smaller peak at ˜1690 cm⁻¹, were both indicative of an antiparallel β-sheet conformation. An increase in temperature resulted in the gradual disappearance of the peak at 1615 cm⁻¹, indicating a clear loss of β-structural content, in agreement with the CD spectra displayed in FIG. 8. The peak around 315 K is consistent with the disintegration of trimers to dimers immediately to monomers.

### Example 5

### Temperature-Dependent UV-CD Spectra of Ac-(AAKA)₄-NH₂.

A backbone hydrogen bond network analysis was implemented to study the orientation of the strands in the trimer aggregates. A sequence indexing system was adopted to avoid the redundancy in labeling the residues. The first strand was defined as the outside strand with more hydrogen bonds. Therefore, the inner strand was always the second strand.

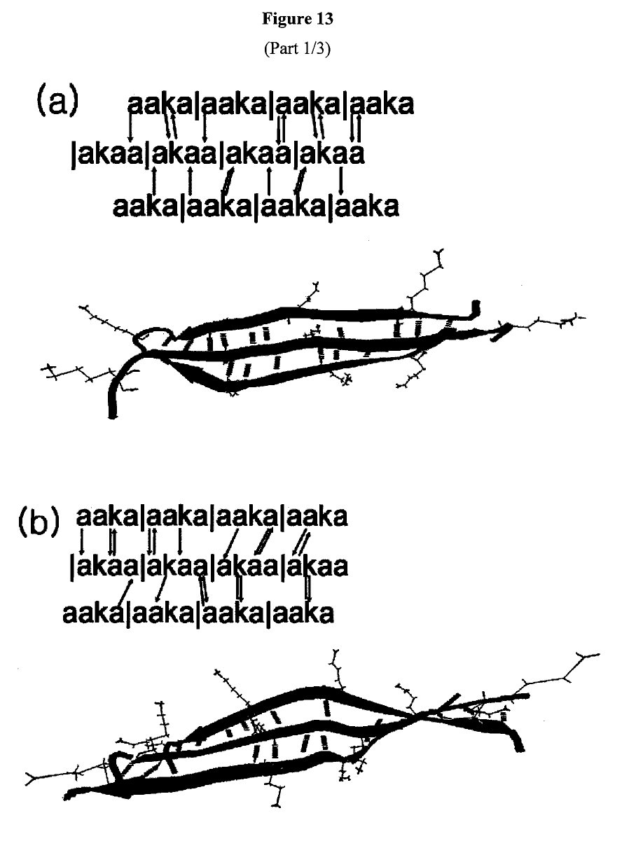

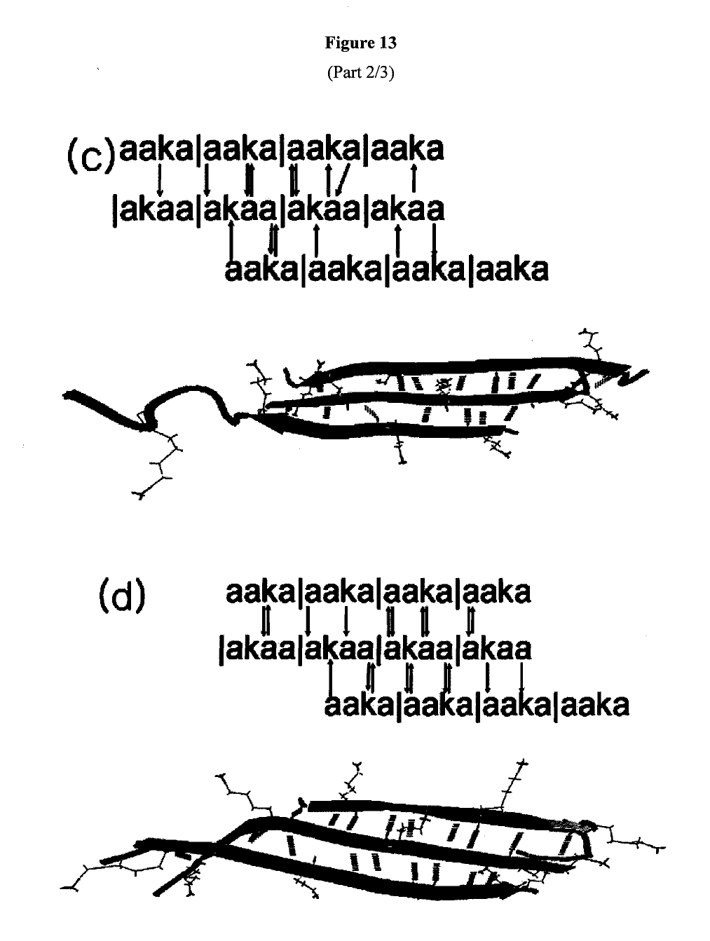

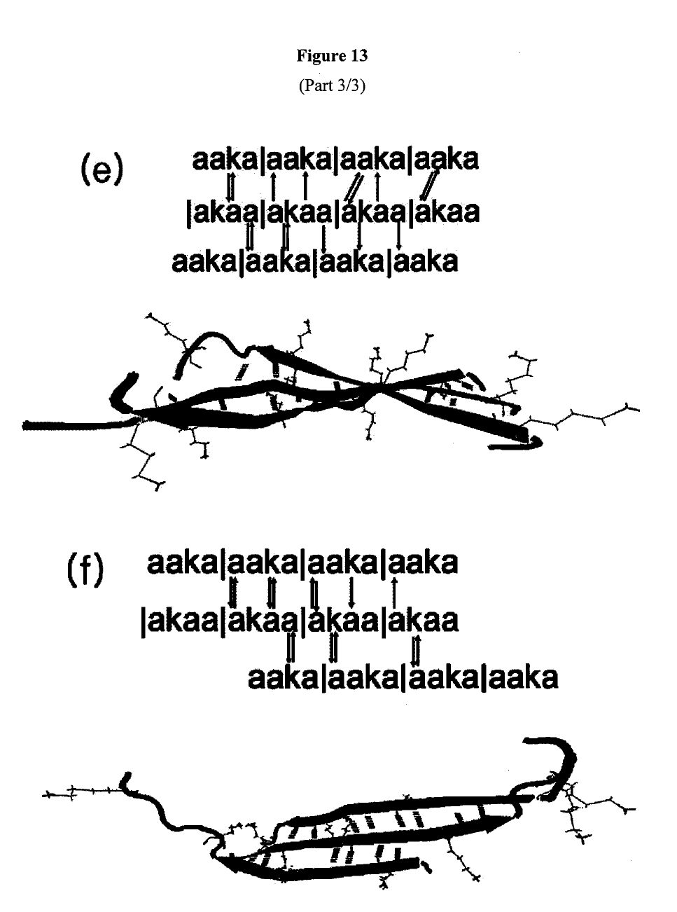

In FIG. 13, the backbone hydrogen bond networks of the selected conformers for the top six most populated clusters are shown. The main structural difference among different clusters was the relative positioning (in register or not) of β-strands. Generally, the conformers with higher energy tended to have more residues at the ends to become random coils. According to the length of the dangling coils at the ends of the strands, due to the misalignment between strands, there were two types of aggregates, namely blunt- and sharp-ended. For example, the third, fourth, and sixth clusters belonged to the sharp ended category, due to the misalignment between second and third strands, while the rest were blunt-ended. Due to the repeat unit and the hydrogen bond patterns, the transition between these clusters may be achieved through interstrand sliding. Once a strand slided a distance of approximately four residues, the hydrogen bond pattern was restored, but the total number of hydrogen bonds might vary. For example, the second strand of the first cluster may slide between the first and third strand to form the conformers of the second and fifth clusters. In the second and fifth clusters, the third strand slided out to form a coil tail and the aggregate transformed into a conformer belonging to the third, fourth and sixth clusters. However, in view of the transition state (FIG. 13C), the interstrand sliding may be achieved by first detaching parts of the strands in the middle or the end, and then sliding in a worm-crawling reptation motion. For example, the representative conformer of the transition state between the free energy well with the lowest free energy and that with the second lowest free energy had the middle section of the third strand detached from the second strand, while the other one had the tail section of the third strand detached from the second strand.

### Example 6

Hydrogel Formation with (AAKA)₄.

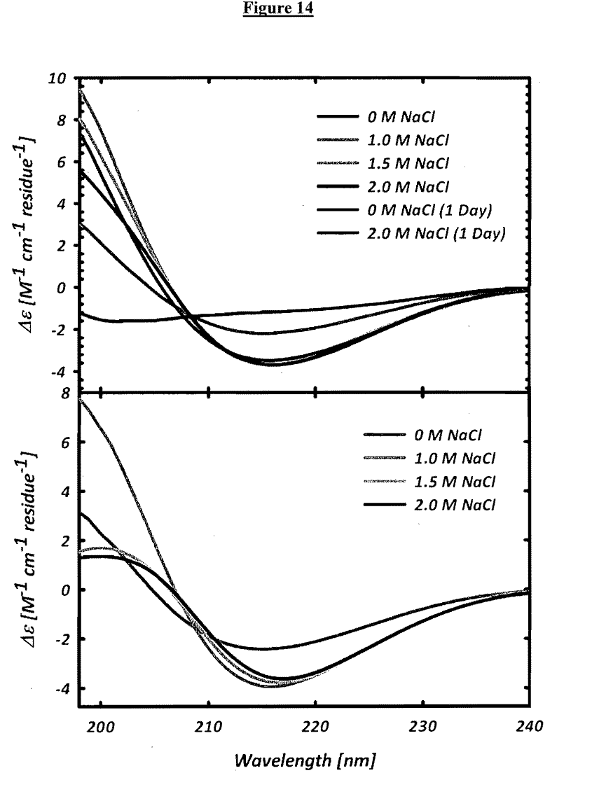

The UV-CD spectra of (AAKA)₄ at concentrations of 3.5 mM and 7.0 mM were recorded at different concentrations of sodium chloride (NaCl). The results are illustrated in FIG. 14. As the concentration of NaCl increased, the negative signal near 215 nm narrowed. This observation suggested that the β-sheet structure was stabilized by the salt present in solution.

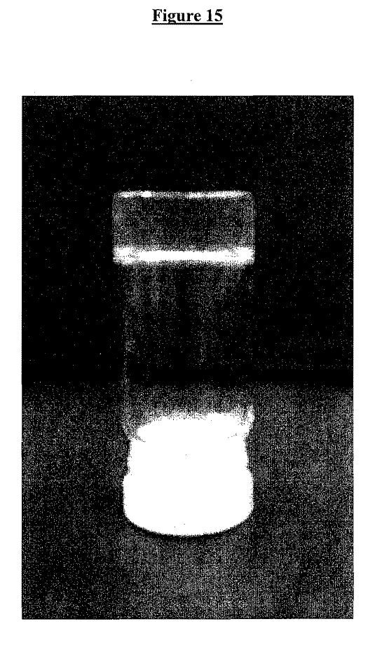

NaCl was added to a freshly aqueous solution of (AAKA)₄, wherein the concentration of peptide was equal to or higher than 0.5 wt %. For final concentrations of NaCl higher than 1.0 M, the n-sheet structure was stabilized, yielding a rigid self-supporting hydrogel. An example of such hydrogel is illustrated in FIG. 15.

### Example 7

### Rheology of (AAKA)₄.

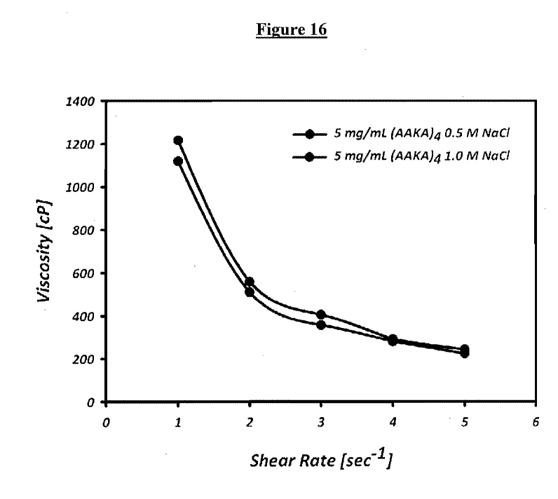

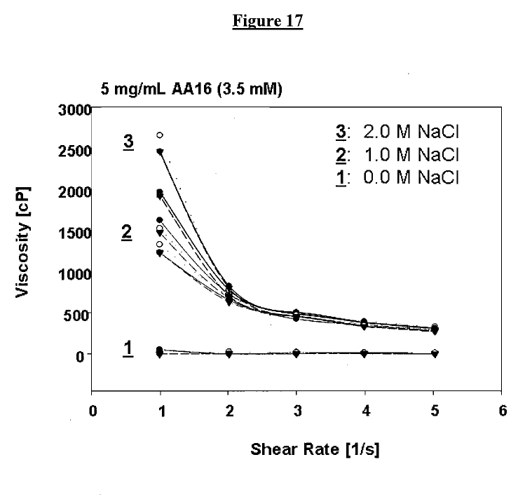

Rheology measurements were made for solutions of (AAKA)₄ (concentration of 5 mg/mL or 3.5 mM) in 0.5 M and 1.0 M NaCl, as shown in FIG. 16. Higher peptide and NaCl concentrations resulted in more viscous gels. The shear-thinning behavior observed in these experiments was consistent with other oligopeptides hydrogels (Schneider, 2006, J. Am. Chem. Soc. 124:15030).

Another rheology experiment is illustrated in FIG. 17, for a solution of (AAKA)₄ (concentration of 5 mg/mL) in 0.0 M, 1.0 M and 2.0 M NaCl. FIG. 17 illustrates three experimental curves for each concentration of NaCl.

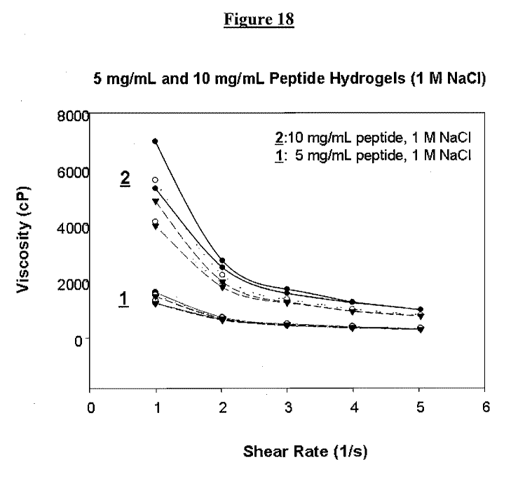

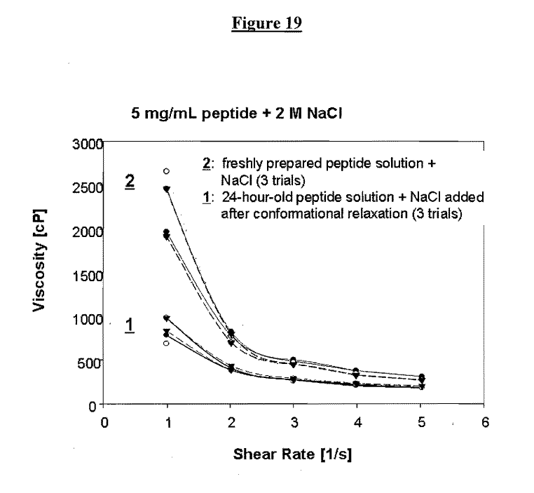

Yet another rheology experiment is illustrated in FIG. 18, for a 5 mg/mL and a 10 mg/mL solution of (AAKA)₄ in 1 M NaCl. FIG. 18 illustrates three experimental curves for each value of concentration of peptide.

As illustrated above, (AAKA)₄ was conformationally unstable upon dissolution in water, prior to the addition of salt and consequent hydrogel formation. When salt was added to the (AAKA)₄ some time after the solution was formed (so that the peptide had enough time to conformationally relax), this resulted in a much less viscous and rigid hydrogel. This is illustrated in FIG. 19, where the viscosity of a 5 mg/mL (AAKA)₄ solution comprising 2 M NaCl was compared with the viscosity of a similar peptide solution wherein NaCl was added after ˜24 hours. This result demonstrates that the viscosity of the (AAKA)₄ solution or equivalent peptide solutions may be modulated by adding varying amounts of salt to the solution at varying time points after generation of the solution.

### Example 8

### Atomic Force Microscopy (AFM).

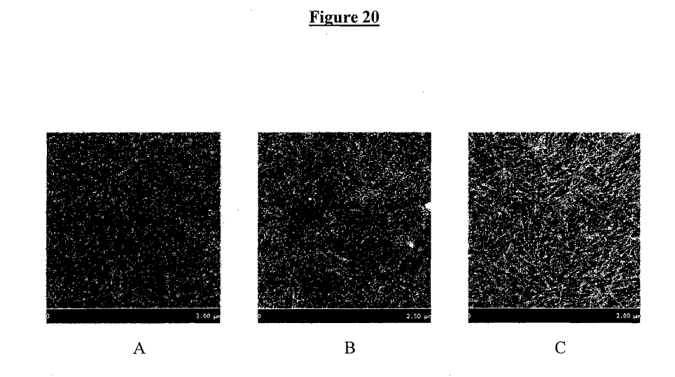

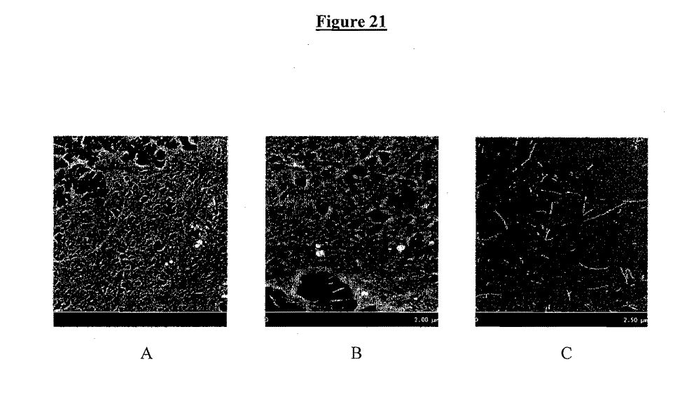

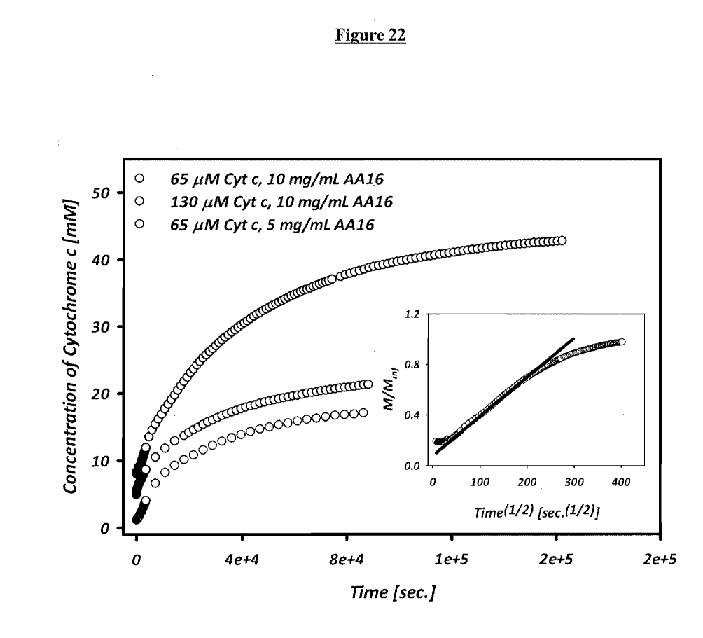

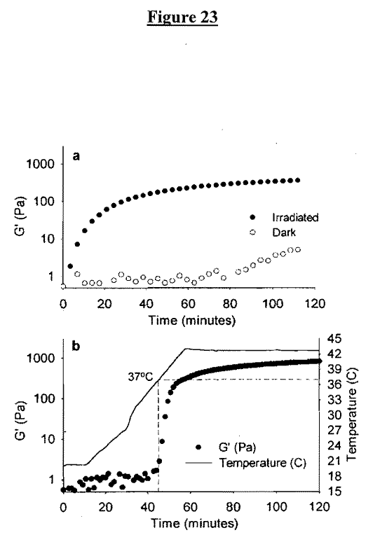

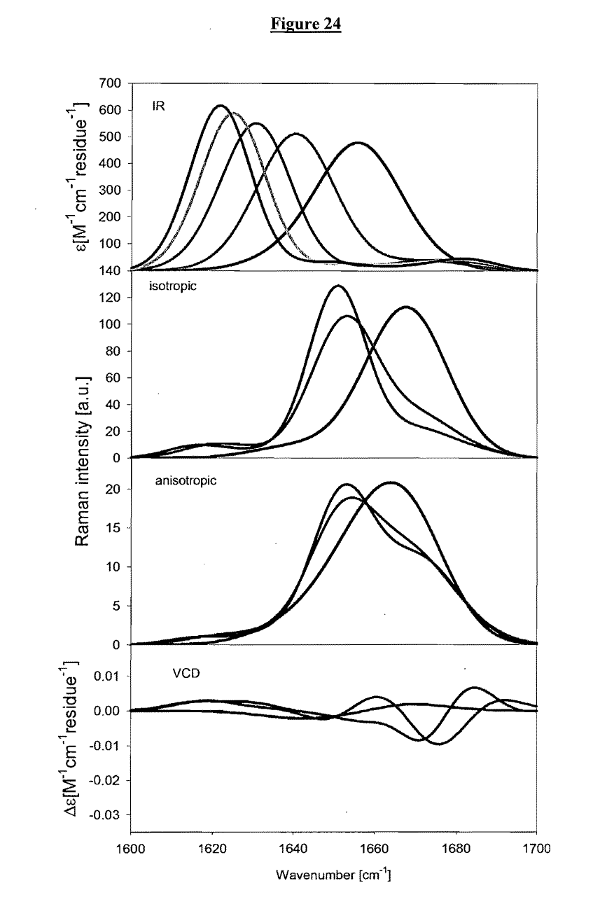

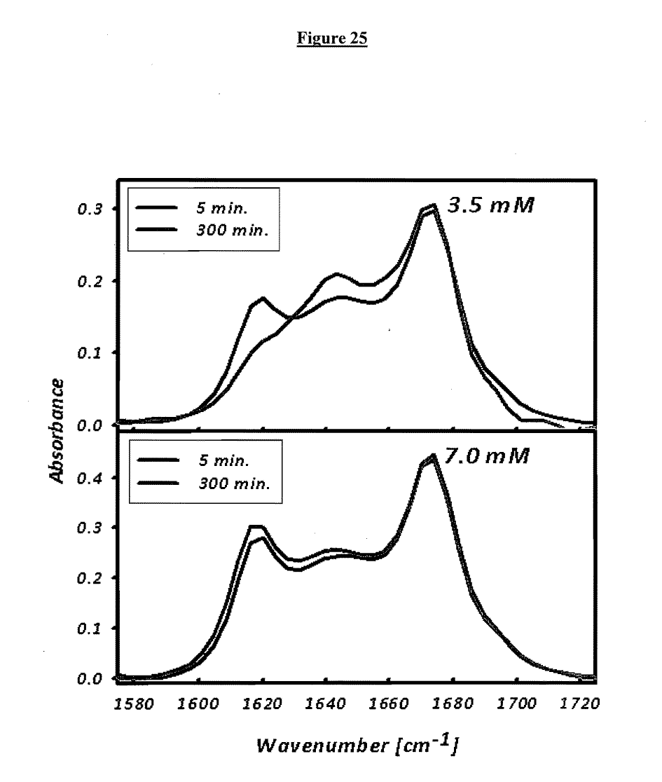

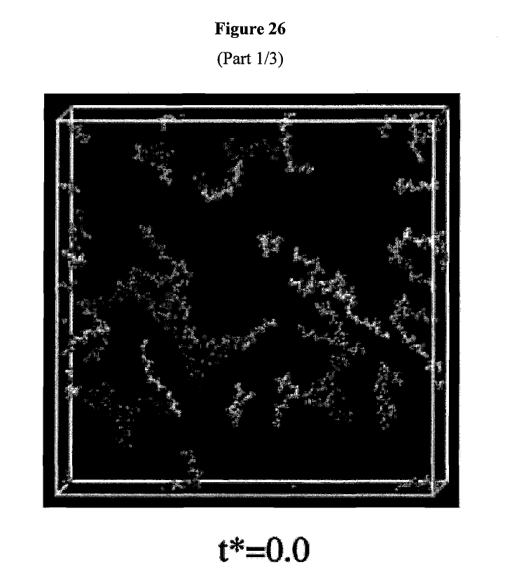

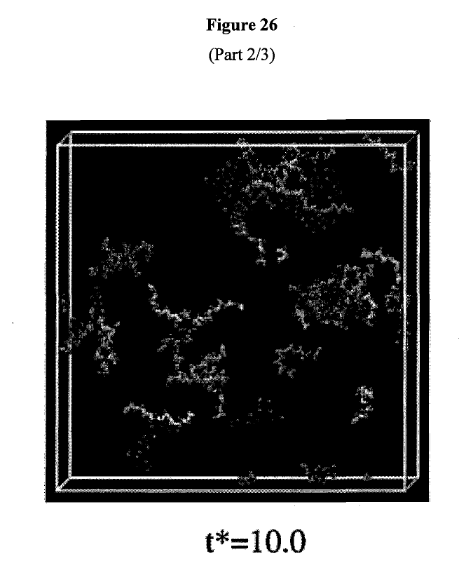

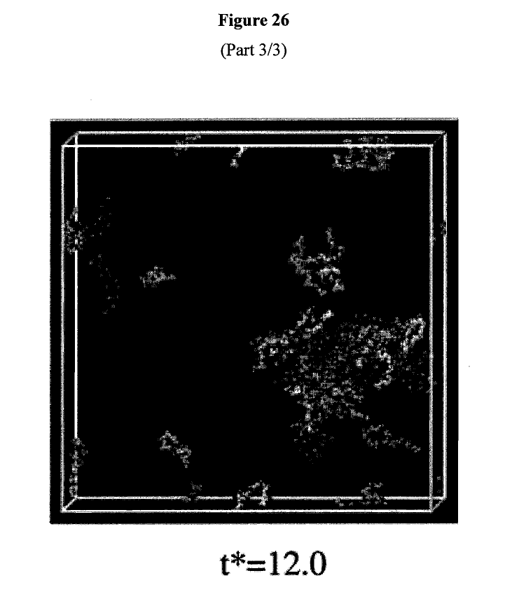

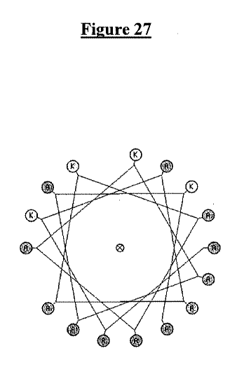

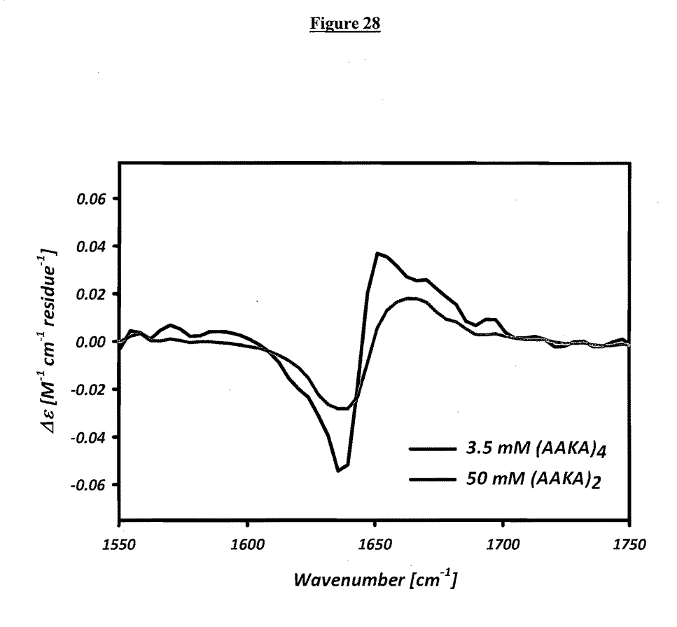

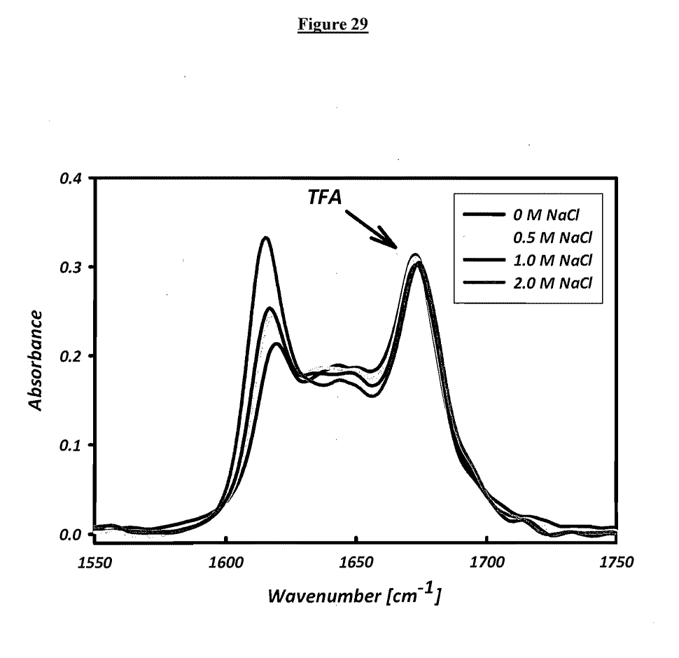

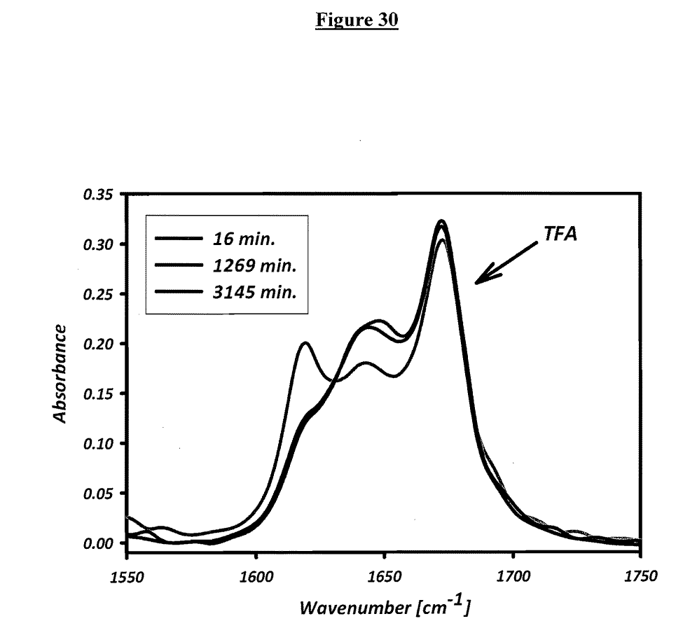

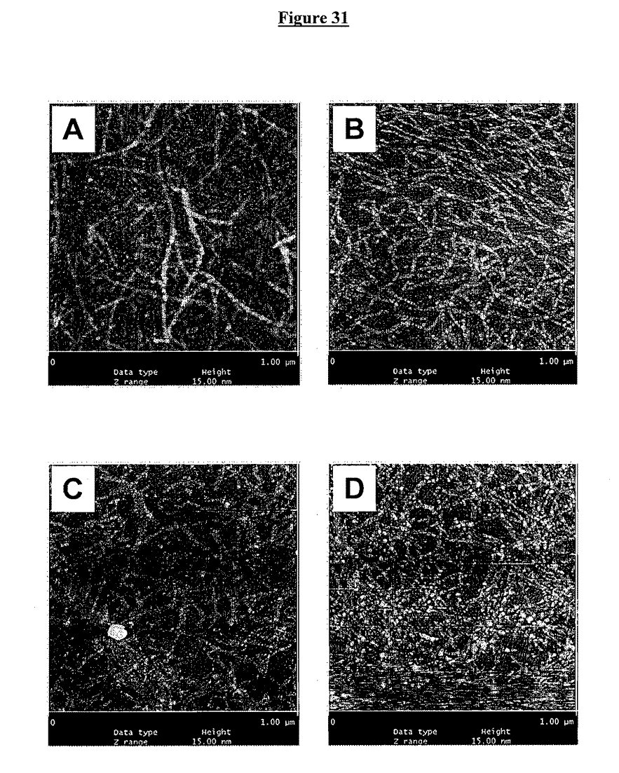

Time-dependent atomic force analysis was performed with a 7 mM solution of (AAKA)₄, as shown in the height images illustrated in FIG. 20. Initially amorphous aggregates were formed (FIG. 20A), with eventual formation of beaded-filamentous structures (FIG. 20B), which eventually stabilized and became more filamentous (FIG. 20C).

As shown in FIG. 21, atomic force analysis further indicated that a web-like architecture was formed upon hydrogel formation in a 7 mM solution of (AAKA)₄ (FIGS. 21A and 21B). Fibril-like structures were also observed at a concentration of 7 μM (FIG. 21C).

### Example 9

Controlled Release using a Hydrogel.

The ability of a peptide-based hydrogel to incorporate and release a molecule was evaluated using equine cytochrome c. A solution of equine cytochrome c was prepared in D₂O and to this solution was added (AAKA)₄. A saturated solution of NaCl in D₂O was then added to yield the desired concentrations of peptide, protein and salt.

In order to follow the release of cytochrome c from the hydrogel matrix, the following protocol was followed. D₂O (500 μL) was placed on top of the hydrogel, and the release of cytochrome c into the upper liquid was monitored by monitoring the UV absorption of the solution at 275 nm. The apparent diffusion coefficient of the protein was determined using the following equation:

Mₜ/M_{∞}=[16.D_{app.t}/(π.H²)]^{0.5}

wherein Dₐₚₚ is the apparent diffusion coefficient, Moo is the mass of protein at infinite time, Mₜ is the mass of protein at time (t), and H is the hydrogel thickness.

The results of the experiments are summarized in Table 1.

TABLE 1
Apparent diffusion coefficients calculated
for cytochrome c in (AAKA)₄ hydrogel.

| [peptide] | Rg | [protein] | [NaCl] | Dₐₚₚ [m²/s] |
|---|---|---|---|---|
| 10 mg/mL | ~1.3 | 65 mM | 1 M | 0.59 ± 0.03 × 10⁻¹⁰ |
| 10 mg/mL | ~1.3 | 130 mM | 1 M | 0.64 ± 0.02 × 10⁻¹⁰ |
| 5 mg/mL | ~1.3 | 65 mM | 1 M | 0.50 ± 0.02 × 10⁻¹⁰ |
| 5 mg/mL | ~1.3 | 130 mM | 1 M | 0.55 ± 0.02 × 10⁻¹⁰ |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of preparing a hydrogel of a defined viscosity, wherein said method comprises the steps of:
dissolving a peptide in an aqueous solution, wherein said peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4), to generate a first solution;
equilibrating said first solution at a given temperature for a given period of time; and,
adding an amount of salt ingredient to said first solution, to generate a second solution, wherein said second solution comprises said hydrogel of said defined viscosity.

2. The method of claim 1, wherein the concentration of said peptide in said second solution ranges from about 1 mg/mL to about 20 mg/mL.

3. The method of claim 2, wherein said concentration ranges from about 5 mg/mL to about 10 mg/mL.

4. The method of claim 1, wherein said given period of time ranges from about 1 minute to about 48 hours.

5. The method of claim 1, wherein said given period of time is about 24 hours.

6. The method of claim 1, wherein said given temperature is about 25° C.

7. The method of claim 1, wherein said salt ingredient comprises sodium chloride.

8. The method of claim 1, wherein the concentration of said salt ingredient in said second solution ranges from about 0.5 M to about 10 M.

9. The method of claim 1, wherein said defined viscosity ranges from about 500 cP to about 3000 cP for a shear rate of 1/s.

10. The method of claim 1, wherein said defined viscosity ranges from about 800 cP to about 2500 cP for a shear rate of 1/s.

11. A method of promoting controlled release of a biomolecule into a medium using a hydrogel of a defined viscosity, wherein said method comprises the steps of:
preparing a first solution comprising said biomolecule;
dissolving a peptide in said first solution, wherein said peptide is selected from the group consisting of (AAKA)₄ (SEQ ID NO:1), Ac-(AAKA)₄ (SEQ ID NO:2), (AAKA)₄-NH₂ (SEQ ID NO:3), and Ac-(AAKA)₄-NH₂ (SEQ ID NO:4), to generate a second solution;
equilibrating said second solution at a given temperature for a given period of time;
adding an amount of salt ingredient to said second solution, to generate a third solution, wherein said third solution comprises said hydrogel of said defined viscosity; and,
contacting said third solution with said medium, whereby said biomolecule undergoes controlled release into said medium.

12. The method of claim 11, wherein the concentration of said peptide in said third solution ranges from about 1 mg/mL to about 20 mg/mL.

13. The method of claim 12, wherein said concentration of said peptide in said third solution ranges from about 5 mg/mL to about 10 mg/mL.

14. The method of claim 11, wherein said given period of time ranges from about 1 minute to about 48 hours.

15. The method of claim 14, wherein said given period of time is about 24 hours.

16. The method of claim 11, wherein said given temperature is about 25° C.

17. The method of claim 11, wherein said salt ingredient comprises sodium chloride.

18. The method of claim 11, wherein the concentration of said salt ingredient in said third solution ranges from about 0.5 M to about 10 M.

19. The method of claim 11, wherein said defined viscosity ranges from about 500 cP to about 3000 cP for a shear rate of 1/s.

20. The method of claim 19, wherein said defined viscosity ranges from about 800 cP to about 2500 cP for a shear rate of 1/s.


SEQUENCE LISTING (8 sequences)

SEQ ID NO: 1 - protein, 16 aa, Artificial Sequence
  chemically synthesized
AAKAAAKAAAKAAAKA

SEQ ID NO: 2 - protein, 16 aa, Artificial Sequence
  chemically synthesized
XAKAAAKAAAKAAAKA

SEQ ID NO: 3 - protein, 16 aa, Artificial Sequence
  chemically synthesized
AAKAAAKAAAKAAAKX

SEQ ID NO: 4 - protein, 16 aa, Artificial Sequence
  chemically synthesized
XAKAAAKAAAKAAAKX

SEQ ID NO: 5 - protein, 12 aa, Artificial Sequence
  chemically synthesized
XAKAAAKAAAKX

SEQ ID NO: 6 - protein, 16 aa, Artificial Sequence
  chemically synthesized
XAXAAAXAAAXAAAXX

SEQ ID NO: 7 - protein, 16 aa, Artificial Sequence
  chemicaly synthesized
FEFEFKFKFEFEFKFK

SEQ ID NO: 8 - protein, 8 aa, Artificial Sequence
  chemically synthesized
AAKAAAKA